(19) United States
(12) Patent Application Publication
(10) Pub. No.: US 2015/0080765 A1
(43) Pub. Date: Mar. 19, 2015

(54) System for Detecting Lameness in Sport Horses and other Quadrupeds

(71) Applicant: Kyle Douglas Lampe, Bellevue, WA (US)

(72) Inventor: Kyle Douglas Lampe, Bellevue, WA (US)

(21) Appl. No.: 14/486,540

(22) Filed: Sep. 15, 2014

Related U.S. Application Data
(60) Provisional application No. 61/877,694, filed on Sep. 13, 2013.

(51) Int. Cl. A01K 29/00; A61B 5/11

(52) U.S. Cl. CPC A01K 29/005; A61B 5/112
USPC 600/595

(57) ABSTRACT

A system that utilizes computerized depth perception to automatically scan physical objects, e.g. a quadruped such as a sport horse, over time thereby enabling a detailed analysis of its movement, and changes thereof over time, without the need for attaching sensors to the body of the horse, or requiring a force plate or high speed expensive cameras is disclosed herein. The system is inexpensive enough that non-specialists, e.g. non-veterinary trained quadruped owners, may install the system at an appropriate location such as a horse barn rather than requiring horses to be transported to a specialist for diagnosis.

### CROSS-REFERENCE TO RELATED APPLICATIONS

The present application claims the benefit of U.S. Provisional Patent Application No. 61/877,694 filed Sep. 13, 2013, entitled “System for Detecting Lameness in Sport Horses and other Quadrupeds,” which is incorporated herein by reference in their entirety as if fully set forth herein.

### FIELD OF THE DISCLOSURE

This disclosure relates generally to the field of detecting lameness is quadrupeds, and more specifically, to a system utilizing computerized depth perception to detect lameness in sport horses and other quadrupeds.

### BACKGROUND

Detecting lameness in sport horses is a significant and challenging task for veterinarians and horse owners. Current methods for detecting subtle lameness in a horse are subjective, and requires a highly skilled vet. Studies have shown that even between vets there is an amount of subjectivity, and question the common practices involved in performing a lameness exam. However delay in detecting an injury in a sport horse will delay treatment, cost more, and possibly hinder the horse's future success.

A veterinary exam and lameness workup is the current main technique for evaluating lameness in horses. The vet watches the horse move at a trot (a symmetrical gait) across hard and soft surfaces, in a straight line and in a circle. The symmetry of the gait allows the vet to try and pick up asymmetry of movement in the horse to pinpoint lameness. However, such diagnosis are prone to being subjective, and when the lameness is subtle studies have shown not all vets will agree.

Automation is a big field of research with lameness diagnostics, to try and add an objective perspective. The most common method used so far involves attaching many sensors (perhaps dozens or up to a hundred) to the horse, then filming the horse trotting with high speed cameras and tracking the sensors movement. Currently the gold standard in automated lameness evaluation involves the animals trotting across force plates to measure how they distribute their weight amongst limbs. However both systems require expensive custom setups which require the horse to be brought to the diagnosis facility and are incapable of remotely diagnosing a horse. They also require placing things on the horse making it a less natural movement.

### SUMMARY

A system that utilizes computerized depth perception to automatically scan physical objects, e.g. a quadruped such as a sport horse, over time thereby enabling a detailed analysis of its movement, and changes thereof over time, without the need for attaching sensors to the body of the horse, or requiring a force plate or high speed expensive cameras is disclosed herein. The system is inexpensive enough that non-specialists, e.g. non-veterinary trained quadruped owners, may install the system at an appropriate location such as a horse barn rather than requiring horses to be transported to a specialist for diagnosis.

Furthermore, a system installed in a horse's own barn can measure those horses over time, even at times when they are healthy. This establishes a baseline that will further help with the lameness diagnosis, as typically veterinarians only examine these horses when they are already lame. Compilations of repeated observations of individual animals, and the posture or altering posture thereof, over extended periods of time, i.e. longitudinal studies of the quadruped movement, may allow earlier and more fine-tuned diagnosis of problems. It may also be used for rehabilitation to quantify when the horse has returned to its healthy movement.

In some embodiments, the System for Detecting Lameness in Sport Horses and other Quadrupeds comprises a computer vision system operable to perceive the depth of a plurality of points on a specimen quadruped and a control system being operable to at least: receive an input from the computer vision system; generate from the input a plurality of three-dimensional representations of the specimen quadruped, wherein at least two of the plurality of three-dimensional representations correspond to a different time from each other; utilize the plurality of three-dimensional representations for the creation of a mathematical representation of a movement of the specimen quadruped; update the mathematical representation of a movement of the specimen quadruped and detect deviations of the representation of the movement over time.

The following embodiments and descriptions are for illustrative purposes only and are not intended to limit the scope of the System for Detecting Lameness in Sport Horses and other Quadrupeds. Other aspects and advantages of the present disclosure will become apparent from the following detailed description.

### BRIEF DESCRIPTION OF THE DRAWINGS

Embodiments of the present disclosure are described in detail below with reference to the following drawings. These and other features, aspects, and advantages of the present disclosure will become better understood with regard to the following description, appended claims, and accompanying drawings. The drawings described herein are for illustrative purposes only of selected embodiments and not all possible implementations and are not intended to limit the scope of the present disclosure. Also, the drawings included herein are considered by the applicant to be informal.

FIG. 1 illustrates each of a quadruped specimen and a mathematical representation of the quadruped specimen in the upper right corner of the figure.

FIG. 2 illustrates an enlarged view of the mathematical representation of the quadruped specimen of FIG. 1.

FIG. 3a is a representation of a field of view of a computer vision system with a quadruped specimen within the field of view.

FIG. 3b illustrates a mathematical representation of the quadruped specimen of FIG. 3a.

### DETAILED DESCRIPTION

A system that utilizes computerized depth perception to automatically scan physical objects, e.g. a quadruped such as a sport horse, over time thereby enabling a detailed analysis of its movement, and changes thereof over time, without the need for attaching sensors to the body of the horse, or requiring a force plate or high speed expensive cameras is disclosed herein.

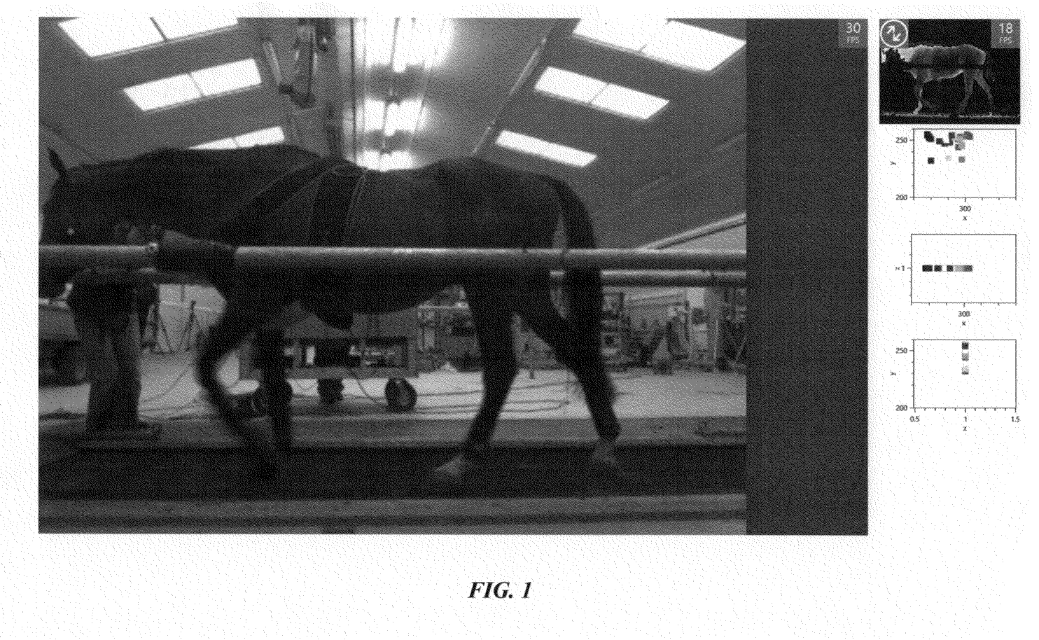

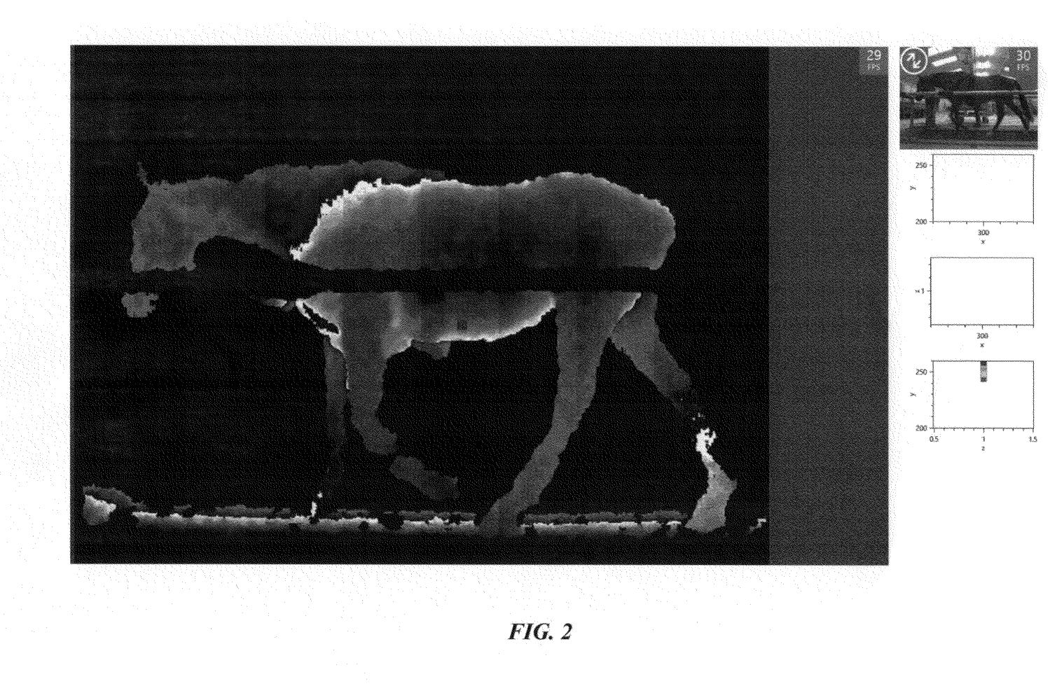

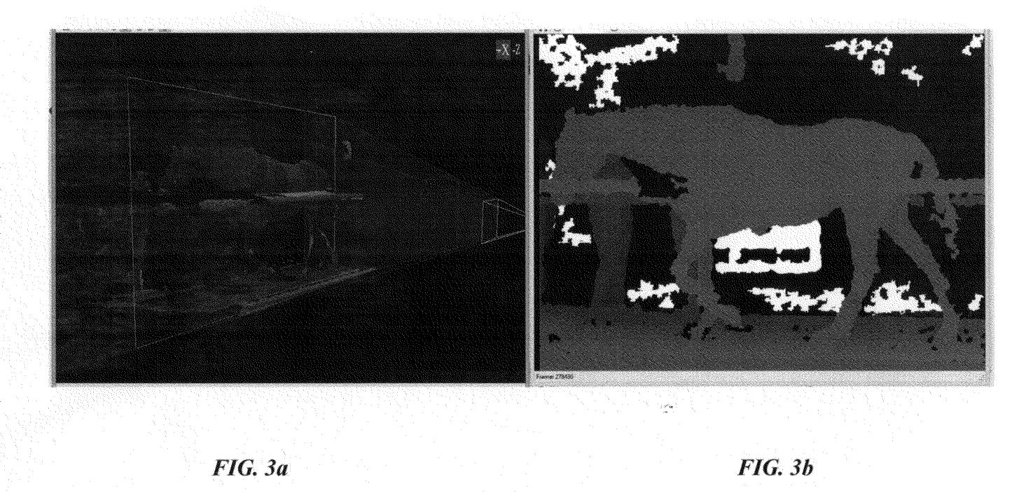

Specific details of certain embodiments are set forth in the following description and in FIGS. 1-3b to provide a thorough understanding of such embodiments. The present System for Detecting Lameness in Sport Horses and other Quadrupeds may have additional embodiments, may be practiced without one or more of the details described for any particular described embodiment, or may have any detail described for one particular embodiment practiced with any other detail described for another embodiment.

In some embodiments, the System for Detecting Lameness in Sport Horses and other Quadrupeds utilizes advanced computer vision depth perception to record longitudinal studies of the quadruped movement. In some embodiments, the system generates one or more point clouds, e.g. a set of data points in a coordinate system representing an object of interest, of individual specimens. For example, the system may generate a point cloud of a specimen thereby enabling the generation of one or more 3 dimensional computer-aided design (CAD) models of the specimen. Use of computer vision has substantial advantages over human vision in that computer vision is able to detect small and subtle deviations in specimen movement, i.e. even changes which would be practically imperceptible to the human observer.

In some embodiments, the system generates multiple point clouds and/or 3D CAD models of the specimen over time, e.g. longitudinal studies, and generates 3D CAD video representations therefrom. Accordingly, such video representations can be played back at a later date to assist in diagnosis by caretakers and/or veterinarians. Such ability has numerous advantages including the ability to playback the generated video footage multiple times as opposed to attempting to re-observe the specimen in real time. Another advantage is the ability to send such video representations electronically, e.g. via email, to interested persons thereby reducing and/or eliminating the need for costly and time-consuming onsite visits.

In some embodiments, the system will utilize the multiple longitudinal point clouds and/or 3D CAD models of the specimen to output objective data to assist in diagnosis by caretakers and/or veterinarians. This objective data is determined by a mathematical analysis of the point clouds. It can encompass much information, which can include but is not limited to: how much weight is borne by individual legs over periods of time, 3D shifts in center of mass, height of certain body parts during movement, and angles of various joints.

Generally, the system creates a baseline of an individual specimen and subsequently and continuously monitors the individual specimens for even slight deviations from the predetermined baseline. For example, the specimen may be a sport horse and a baseline may be created at a time when the sport horse is known to be healthy. By monitoring the particular sport horse continuously for even slight deviations from the baseline, the system detects even subtle deviations in gait, or other posture or movement characteristic, to alert caretakers before any potential injury becomes exacerbated by continued exertion and/or lack of attention and treatment.

In some embodiments, the System for Detecting Lameness in Sport Horses and other Quadrupeds is configured to alert one or more predetermined interested parties upon the detection of a predetermined event, e.g. a deviation from an individual specimen's healthy baseline gait. For example, the system may be configured to automatically transmit email alerts including generated 3D CAD video representations of particular specimens upon a presumed deviation in gait. The alerted parties may then take appropriate action as required.

In some embodiments, the system is configured to permanently store all data collected on designated specimens. Furthermore, in preferred embodiments the system individually identifies specimens thereby eliminating the need for manual input of specimen identification by caretakers. For example, in some embodiments the system identifies particular specimens through comparison of current data input, e.g. real time computer vision depth perception sensory input, with previously collected and/or uploaded data associated with the same specimen. In some embodiments, the system identifies particular specimens through use of Radio-frequency identification (RFID), e.g. an RFID tag may be attached to or implanted within each individual specimen. Any other suitable type of identification method may also be used without departing from the spirit and scope of the System for Detecting Lameness in Sport Horses and other Quadrupeds.

In some embodiments, the system enables individual animal owners, veterinarians, and/or other caretakers to retrieve information about the animal. This information is then displayed in a way that allows either a point in time or longitudinal view of the data stored in the system. This retrieval and display of the accumulated data, analysis, and diagnosis can be used to better understand the history and current state of a horse.

While preferred and alternate embodiments have been illustrated and described, as noted above, many changes can be made without departing from the spirit and scope of the System for Detecting Lameness in Sport Horses and other Quadrupeds. Accordingly, the scope of the System for Detecting Lameness in Sport Horses and other Quadrupeds is not limited by the disclosure of these preferred and alternate embodiments. Instead, the scope of the System for Detecting Lameness in Sport Horses and other Quadrupeds should be determined entirely by reference to the claims. Insofar as the description above and the accompanying drawings (if any) disclose any additional subject matter that is not within the scope of the claims below, the inventions are not dedicated to the public and Applicant hereby reserves the right to file one or more applications to claim such additional inventions.

What is claimed is:

1. A gait detection apparatus, comprising:
a computer vision system operable to perceive the depth of a plurality of points on a specimen quadruped;
a control system being operable to at least:
receive an input from the computer vision system;
generate from the input a plurality of three-dimensional representations of the specimen quadruped, wherein at least two of the plurality of three-dimensional representations correspond to a different time from each other;
utilize the plurality of three-dimensional representations for the creation of a mathematical representation of a movement of the specimen quadruped;
update the mathematical representation of a movement of the specimen quadruped and detect deviations in the representation of the movement over time.
